(19) United States
(12) Patent Application Publication
(10) Pub. No.: US 2009/0099215 A1
(43) Pub. Date: Apr. 16, 2009

(54) Metabolic enhancing properties of norphenephrine and its salts

(76) Inventors: Derek W. CORNELIUS, Cape Girardeau, MO (US); Gary L. HAYNES, Scott City, MO (US)

Correspondence Address: Darby & Darby (Formerly Michael J. Keller), P.O. Box 770, Church Street Station, New York, NY, 10008-0770, US

(21) Appl. No.: 11/974,031

(22) Filed: Oct. 11, 2007

(51) Int. Cl. A61K 31/135; A23L 1/29; A61K 31/437; A61K 31/522; A61P 25/00; A61P 3/00; A61P 3/04

(52) U.S. Cl. 514/263.31; 426/648; 514/299; 514/653

(57) ABSTRACT

Norphenephrine and its salts are disclosed which have powerful metabolic enhancing effects in humans via oral administration. The metabolic enhancing properties include positive blood sugar regulation, increased thermogenesis and fat loss, increased anticatabolism/anabolism, decreased appetite, improved mental focus and psychological outlook and increased energy production and anti-fatigue actions.

### BACKGROUND OF THE INVENTION

Norphenephrine is a naturally occurring trace amine that occurs throughout the animal kingdom. It is found in invertebrates such as insects and arthropods and in vertebrates including mammals. In mammals, it is found in organs such as brain and nervous tissue. The widespread nature of this naturally occurring amine translates into it being consumed in the normal human diet, albeit in small quantities.

Recently, it has been discovered by the inventors that norphenephrine and its salts have specific and beneficial metabolic properties upon oral administration. The metabolic enhancing properties include positive blood sugar regulation, increased thermogenesis and fat loss, increased anticatabolism /anabolism, decreased appetite, improved mental focus and psychological outlook and increased energy production and anti-fatigue actions. The inventors found that the compounds of this invention are in many ways similar to amphetamines in that they decrease appetite, reduce fatigue, increase energy, improve mental outlook and increase thermogenesis. The difference, however, is that norphenephrine and the salts of this invention are not addictive like amphetamines. This is a significant improvement since the addictive nature of amphetamines and their derivatives is the single biggest obstacle to their widespread use. The inventors discovered that norphenephrine and its salts are not addictive, or even habituating, even at high dosages. Additional improvements over amphetamines are that these norphenephrine compounds improve blood sugar regulation and have an anti-catabolic/anabolic effect on proteinaceous tissues such as the muscles.

Prior art demonstrates only a few physiological effects or uses of norphenephrine in humans. A main use of norphenephrine has been to increase blood pressure in hypotensive situations or conditions. Norphenephrine has proven itself to be a reliable treatment in these conditions and has been used as such for many years. Another use of norphenephrine has been to improve urinary incontinency. Again, norphenephrine has proven quite useful for this condition and has been used successfully for many years in this capacity.

The inventors have found that norphenephrine and its salts specifically enhance the loss of adipose tissue while preserving lean body mass such as muscle tissue. A significant problem for anyone trying to lose unwanted adipose tissue is the concurrent loss of lean body mass. Lean body mass includes tissues such as muscle, kidney, liver, heart and bone. For a person to feel healthy and strong, these tissues must also be healthy and strong. If, while dieting or taking a weight reducing agent, lean body mass is decreased, it has an immediate affect on the overall health and well-being of the organism. Norphenephrine used within the scope of this invention preserves the precious lean body mass at the expense of adipose tissue. The effect is what is known as nutrient partitioning where nutrients are preserved or partitioned into lean body mass and expended or metabolically burned in adipose tissue.

This invention also discloses that norphenephrine and it salts can be used as weight reducing agents either with or without a calorie reducing diet or exercise. Although any weight reducing program works better when calories are restricted and aerobic exercise is performed, we have found that this is not necessary. Norphenephrine and its salts cause a noticeable and quantifiable decrease in fat mass even with making no changes to a user's current diet and exercise regimen.

It is also disclosed that norphenephrine and its salts increase a user's overall metabolic rate. Thus, although a specific adipose reducing effect is achieved from the nutrient partitioning effect, norphenephrine also actually causes an increase in the amount of calories that are burned or expended. By increasing the overall metabolic rate, fatigue is signficantly reduced and perceived energy and ability to accomplish vigorous work related tasks is amplified.

It is also disclosed that norphenephrine and its salts reduce the appetite for food of the user. Although user variability exists, generally speaking this effect is quite pronounced and strong. Within 30 minutes, the user experiences a vastly reduced appetite for food.

A further disclosure is that norphenephrine and its salts have a sustained and noticeable effect on a person's focus and acuity as well as a person's psychological outlook. Persons consuming norphenephrine in quantities disclosed herein report anti-depressive properties and experience a positive attitude about life. Furthermore, following uptake of norphenephrine, subjects report an increase in the ability to focus and concentrate on difficult tasks such as test taking and problem solving.

Finally, the inventors have discovered that norphenephrine and its salts are potent agents in regulating blood sugar and insulin levels. We have found that they specifically increase insulin sensitivity in muscle tissue apart from adipose tissue. This may help explain the nutrient partitioning effects of this invention. Insulin is an anabolic hormone to all tissues. Thus, increasing the ratio of the effectiveness of insulin in muscle as compared to adipose tissue would practically help a user preserve or increase muscle mass while decreasing fat mass.

We have found that a wide range of organic and inorganic norphenephrine salts conform to the scope of this present invention. The range of salts which conforms to this invention includes, but is not limited to, inorganic salts such as hydrochloride, hydrobromide, sulfate, phosphate, nitrate, carbonate, hydroselenate, metasilicate, permanganate, etc. Organic salts likewise have been shown to conform to the scope of this invention and include but are not limited to carboxylic acids such as citrate, malate, formate, tartrate, maleate, fumarate, tartarate, picolinate, nicotinate, pyruvate, succinate, glycinate, glutamate, ascorbate, decanoate, palmitate, oleate, acetate, propionate, etc. In respect to di-, tri- and tetra- carboxylic acids, the scope of this invention includes dinorphenephrine, trinorphenephrine and tetranorphenephrine carboxylic salts. Finally, other organic salts such as organic sulfonic, carbonic, and phosphonic salts are included in the scope of this patent. Examples of these are methanesulfonate, isethionate, toluenesulfonate, ethylcarbonate, methylcarbonate, etc.

Human dosing of norphenephrine and its salts is preferable in the range of 5 mg-2500 mg per day calculated as base norphenephrine. An even more preferable dosage is between 25 mg-250 mg per day. Although norphenephrine and its salts can be dosed once per day for a desired effect, we have found that it is best to divide the dosage into two or three equal amounts given eight to twelve hours apart. This helps to ensure steady blood values and an overall more powerful, consistent effect. Norphenephrine may be administered in the form of tablets, capsules, sachets, effervescent powder or tablets, softgels, liquid or other oral delivery system known in the art in both immediate and controlled release forms.

It has also been discovered that the co-administration of methylxanthines as an enhancing agent with norphenephrine and its salts increases the activity of norphenephrine. Specifically, the activity is increased when combined with caffeine, theobromine, paraxanthine or theophylline in specific ratios. Any ratio of a methylxanthine to norphenephrine within the range of 1:4 to 3:1 will be effective as long as it is consistent with the effective dosage ranges for norphenephrine as disclosed herein. However, there are some practical limitations on dosing a combination product comprising a methylxanthine and norphenephrine in terms of safety. For instance, daily doses of caffeine should not exceed 1500 mg because of safety issues. Thus, the ratios above are valid as long as the caffeine does not exceed 1500 mg. Practically, with a daily dosage of norphenephrine of 5 mg-2500 mg, the caffeine dosage can range from about 1 to 1500 mg. The 3:1 ratio is only possible when the norphenephrine is dosed below 375 mg per day.

Why methylxanthines such as caffeine and theobromine increase the metabolic enhancement of norphenephrine is not known conclusively at this time. Caffeine is historically known for activating and increasing the strength of a wide variety of compounds. From aspirin to ephedrine, caffeine is combined for a more potent effect. Without attempting to limit the mechanisms at work, it is the inventors' opinion that caffeine is interfering with the catabolism in the body of norphenephrine thus allowing for greater circulating blood values of this important chemical. It is possible that caffeine is somehow interfering with MAO-B or possibly affecting the metabolism of norphenephrine in some other, important way that is unknown at this time. Regardless of the mechanism, synergy between caffeine and norphenephrine is observed.

Norphenephrine and its salts can also be combined with one or more of the following compounds: yohimbine, forskolin, phenyethylamine, green tea extract, guggul extract or guggulsterones, bergenin, p-octopamine, and evodiamine.

Within the scope of this invention, norphenephrine and its salts may be obtained through natural or synthetic means as long as the dosage meets the minimum amount necessary to cause the desired effect. For instance, norphenephrine is produced as a synthetic compound that can easily be obtained as a food grade chemical to be administered as a food supplement or as a drug. Likewise, extracts of various animals such as snails could be obtained with varying degrees and standardizations of norphenephrine in them.

Those skilled in the art will appreciate that numerous changes and modifications may be made to the preferred embodiments of the invention and that such changes and modifications may be made without departing from the spirit of the invention. It is therefore intended that the appended claims cover all such equivalent variations as fall within the true spirit and scope of the invention.

We claim:

1. A food or food supplement comprising an effective amount of norphenephrine or a bioavailable norphenephrine salt.

2. A food or food supplement according to claim 1 further comprising at least one agent for enhancing the effectiveness of norphenephrine.

3. The food or food supplement of claim 2 wherein the enhancing agent is a methylxanthine.

4. The food or food supplement of claim 3 wherein the enhancing agent is caffeine, theobromine, paraxanthine or theophylline.

5. The food or food supplement of claim 2 wherein the methylxanthine and norphenephrine are present in a ratio from about 1:4 to about 3:1.

6. The food or food supplement of claim 1 in which the effective amount of norphenephrine is between 5 mg and 2500 mg.

7. The food or food supplement of claim 6 wherein the effective amount of norphenephrine is between 25 and 250 mg.

8. The food or food supplement of claim 1 wherein the norphenephrine salt is an inorganic salt.

9. The food or food supplement of claim 8 wherein the norphenephrine salt comprises at least one of the following: hydrochloride, hydrobromide, sulfate, phosphate, nitrate, carbonate, hydroselenate, metasilicate, permanganate, sulfonate, or phosphonate.

10. The food or food supplement of claim 1 wherein the norphenephrine salt is an organic salt.

11. The food or food supplement of claim 10 wherein the norphenephrine salt comprises at least one carboxylate salt.

12. The food or food supplement of claim 11 wherein the carboxylate salt comprises at least one of the of the following: citrate, malate, format, tartrate, maleate, fumarate, tartarate, picolinate, nicotinate, pyruvate, succinate, glycinate, glutamate, ascorbate, deconaoate, palmitate, oleate, acetate or propionate.

13. The dosage form of claim 10 wherein the organic salt comprises at least one of the following: an organic sulfonic, an organic carbonic or an organic phosphonic.

14. The food or food supplement of claim 13 wherein the organic salt comprises at least one of the following: methanesulfonate, isothionate, toluenesulfonate, ethylcarbonate, or methylcarbonate.

15. A method of inducing a physiologic effect selected from the group consisting of weight loss, increased anabolic activity, reduced catabolic activity, increased lean body mass, positive mental outlook or mental acuity, comprising the administration of an effective amount of norphenephrine or a bioavailable norphenephrine salt to a person in need thereof.

16. The method of inducing weight loss according to claim 15 in which the appetite is decreased.

17. The method of inducing weight loss according to claim 15 in which the administration of the norphenephrine or norphenephrine salt induces increased thermogenesis.

18. The method of inducing weight loss according to claim 15 in which the administration of the norphenephrine or norphenephrine salt increases insulin sensitivity and lowers mean blood sugar values.

19. The method of inducing weight loss according to claim 15 in which the weight loss is predominantly from adipose tissue and not from lean body mass.

20. The method of inducing weight loss according to claim 15 in which the effective amount of norphenephrine is between 5 mg and 2500 mg.

21. The method of claim 20 wherein the effective amount of norphenephrine is between 25 and 250 mg.

22. The method of claim 15 wherein the norphenephrine salt is an inorganic salt.

23. The method of claim 22 wherein the norphenephrine salt comprises at least one of the following: hydrochloride, hydrobromide, sulfate, phosphate, nitrate, carbonate, hydroselenate, metasilicate, permanganate, sulfonate, or phosphonate.

24. The method of claim 15 wherein the norphenephrine salt is an organic salt.

25. The method of claim 24 wherein the norphenephrine salt comprises at least one carboxylate salt.

26. The method of claim 25 wherein the carboxylate salt comprises at least one of the of the following: citrate, malate, formate, tartrate, maleate, fumarate, tartarate, picolinate, nicotinate, pyruvate, succinate, glycinate, glutamate, ascorbate, deconaoate, palmitate, oleate, acetate or propionate.

27. The method of claim 24 wherein the organic salt comprises at least one of the following: organic sulfonic, organic carbonic, or organic phosphonic.

28. The method of claim 15, further comprising the administration of an enhancing agent for norphenephrine.

29. The method of claim 28 wherein the enhancing agent is a methylxanthine.

30. The method of claim 29 wherein the methylxanthine is caffeine, theobromine, paraxanthine or theophylline.

31. The method of claim 30 wherein the methylxanthine and norphenephrine are present in a ratio from about 1:4 to about 3:1.
